(19) United States
(12) Patent Application Publication
(10) Pub. No.: US 2015/0272773 A1
(43) Pub. Date: Oct. 1, 2015

(54) INTRAORAL APPARATUS FOR MANAGING SNORING AND OBSTRUCTIVE SLEEP APNEA

(71) Applicants: Hector Rico, Suwanee, GA (US); Mayoor Patel, Suwanee, GA (US)

(72) Inventors: Hector Rico, Suwanee, GA (US); Mayoor Patel, Suwanee, GA (US)

(73) Assignee: MAP Laboratory LLC, Buford, GA (US)

(21) Appl. No.: 14/230,268

(22) Filed: Mar. 31, 2014

(51) Int. Cl. A61F 5/56

(52) U.S. Cl. CPC A61F 5/566

(57) ABSTRACT

A removable, adjustable intraoral apparatus having an upper plate and a lower plate, which, upon insertion into a user's mouth, cause advancement of the lower jaw relative to the upper jaw. One of either the lower plate or the upper plate can include length-adjustable blocks on the occlusal (biting) side thereof for causing further advancement of the lower jaw. Each length-adjustable block includes an advancement screw which is embedded in and extends from one of a pair of stationary raised block portions, and each of the plates without the embedded advancement screws also has a pair of raised block portions extending therefrom. The pairs of upper and lower blocks include contact surfaces which cooperate with each other to exert opposing forces upon the upper and lower dental arches and cause mandibular advancement. The length-adjustable portions allow for further mandibular advancement.

### FIELD OF THE INVENTION

This invention relates to the fields of oral appliances and dentistry and more particularly to a wearable and adjustable intraoral apparatus for relieving upper airway disorders and for the treatment of snoring and sleep apnea.

### BACKGROUND OF THE INVENTION

Some of our most fundamental reflexes involve breathing and maintaining a patent airway. Upper airway disorders in patients can result in a variety of difficulties, such as obstructive sleep apnea, snoring, labored breathing, oxygen starvation, and the resulting physical impairments arising from such disorders, such as the impairment of swallowing and speaking. The pathogenesis of airway obstruction that characterizes upper airway disorders can include both anatomic and functional abnormalities of the upper airway that result in increased air flow resistance. Such abnormalities may include narrowing of the upper airway due to suction forces created during inspiration, the effect of gravity pulling the tongue back to appose the pharyngeal wall, and insufficient muscle tone in the upper airway dilator muscles, among others. It is also believed that excessive soft tissue in the anterior and lateral neck, as commonly observed in obese persons, can apply sufficient pressure to internal structures to narrow the upper airway and restrict air flow.

The tongue is normally maintained in a somewhat forward position under autonomic nervous control, which in turn is effected by the medulla of the brain. This autonomic control may not be maintained in a person who is sleeping, semi-conscious, unconscious or under heavy sedation. If one's head is oriented with the mouth pointing upwards, if the tongue should become too relaxed it can fall back towards the throat, reducing or even closing the airway. If a severe closure condition is not corrected within several minutes, this can reduce oxygen saturation in the brain, and, if left uncorrected, lead to irreversible brain damage.

Severe sleep-related breathing disorders may require positive airway pressure (PAP) therapy. Less severe sleep-related breathing disorders, however, may be treated with other therapies or devices, such as an oral appliance. Generally, an oral appliance includes upper and lower dental plates coupled together in such a way as to impart forward advancement of the user's mandible (i.e., lower jaw) relative to the user's maxilla (upper jaw). Accordingly, an oral appliance may also be referred to as a “mandibular advancement device” or “MAD”. Forward advancement of the mandible helps prevent the soft tissue of the tongue and the throat from collapsing into, and thus blocking, the user's airway during sleep.

Achieving the correct amount of forward mandibular advancement with an oral appliance is important. A user, for example, may continue to suffer from a sleep-related breathing disorder if the forward mandibular advancement imparted by the oral appliance is inadequate (e.g., the soft tissue of the user's tongue and throat are not prevented from collapsing into the user's airway). On the other hand, if the forward mandibular advancement imparted by the oral appliance is excessive, the user may experience unnecessary discomfort. The discomfort may cause undesirable arousal from sleep and/or cause the user to stop wearing the oral appliance altogether.

Some oral appliances include mechanisms for adjusting the amount of mandibular advancement provided by the oral appliance. These and similar oral appliances, however, typically include adjustment mechanisms such as advancement screws either on the cheek side or in the front of the device. When the adjustment mechanism is located on the cheek side, patients may experience sores and discomfort on the inside of their cheeks and in their gums. In designs that have the advancement mechanism in the front of such devices there is limited room for the user's tongue, excessive salivation and soreness of the teeth, lips and gums can also be a problem. Also, these and similar oral appliances typically have adjustment mechanisms that are difficult to operate and tend to be bulky, thereby adversely impacting the user's comfort level.

Thus, while prior art devices for treating snoring and related airway disorders may be useful for their intended purposes, there remains a need for providing an improved intraoral apparatus for relieving upper airway disorders and related problems in which the advancement mechanism does not cause soreness or take up too much intraoral space. A need also exists for an improved oral appliance for the treatment of snoring and sleep apnea in which the advancement mechanism is not located on a side between the teeth and the cheek, or in the front of the device.

### SUMMARY OF THE INVENTION

In light of the foregoing, the present invention provides a removable and adjustable apparatus for advancing the position of the user's lower jaw to correct upper airway disorders. The advancement apparatus includes an adjustable bite block with an advancement screw embedded in acrylic on the biting (occlusal) side of the user's teeth. The adjustable apparatus is intended to help further advance the user's lower jaw in an ongoing fashion, without the need to make multiple devices and without causing soreness or pain in the sides or front of the user's mouth.

A first aspect of the invention provides a removable, adjustable intraoral advancement apparatus for the treatment of obstructive sleep apnea and/or snoring, comprising: (a) a lower plate manufactured to conform to a user's lower dental arch, the lower plate including a pair of lower blocks extending upwardly therefrom; and (b) an upper plate manufactured to conform to the user's upper dental arch, the upper plate including a pair of upper blocks extending downwardly therefrom, wherein the upper and lower blocks include contact surfaces which cooperate with each other to exert opposing forces upon the upper and lower dental arches and cause mandibular advancement, and wherein each of either the lower blocks or the upper blocks comprises a stationary portion and a length-adjustable portion for causing further mandibular advancement, the length-adjustable portions being located on the occlusal side of the user's teeth.

A second aspect of the invention provides a removable, adjustable intraoral advancement apparatus for managing snoring and obstructive sleep apnea, comprising: (a) a lower plate manufactured to conform to a user's lower dental arch, the lower plate including a pair of lower blocks extending upwardly therefrom, each lower block including a stationary portion and a length-adjustable portion; and (b) an upper plate manufactured to conform to the user's upper dental arch, the upper plate including a pair of upper blocks extending downwardly therefrom, wherein the upper and lower blocks include contact surfaces which cooperate with each other to exert opposing forces upon the upper and lower dental arches and cause mandibular advancement, and wherein the length-adjustable portions allow for further mandibular advancement, the length-adjustable portions being located on the occlusal side of the user's teeth.

A third aspect of the invention provides a removable, adjustable intraoral advancement apparatus for managing snoring and obstructive sleep apnea, comprising: (a) a lower plate manufactured to conform to a user's lower dental arch, the lower plate including orthodontic wiring and a pair of lower blocks extending upwardly therefrom; and (b) an upper plate manufactured to conform to the user's upper dental arch, the upper plate including orthodontic wiring and a pair of upper blocks extending downwardly therefrom, each upper block including a stationary portion and a length-adjustable portion, wherein the upper and lower blocks include contact surfaces which cooperate with each other to exert opposing forces upon the upper and lower dental arches and cause mandibular advancement, and wherein the length-adjustable portions allow for further mandibular advancement, the length-adjustable portions being located on the occlusal side of the user's teeth.

The nature and advantages of the present invention will be more fully appreciated from the following drawings, detailed description and claims.

### BRIEF DESCRIPTION OF THE DRAWINGS

A more complete appreciation of the present invention and many of the principles and attendant advantages thereof will be readily understood by reference to the following detailed description when taken in conjunction with the accompanying drawings, in which:

FIG. 1 is a perspective view of one embodiment of the invention which comprises upper and lower plates.

FIG. 2 is a perspective view of an expansion screw assembly that can be used with the present invention.

FIG. 3 is a perspective view showing the upper and lower plates of another embodiment of the invention.

FIG. 4 is a perspective view of an embedded advancement screw with its advancement arm being engaged by a key for lengthening of the advancement screw.

### DETAILED DESCRIPTION OF THE INVENTION

### Definitions:

The term “mandibular advancement” as used herein refers to the anterior movement of a user's mandible (lower jaw) relative to the user's maxilla (upper jaw).

The term “fixed” as used herein means that a particular appliance is installed in such a manner so that the user/patient will not be able to readily remove the appliance, but so that the appliance may be removed by the dental practitioner utilizing the proper tool(s).

The term “removable” as used herein means that a particular appliance is installed in such a manner so that the appliance may be readily removed by both the user/patient and the dental practitioner.

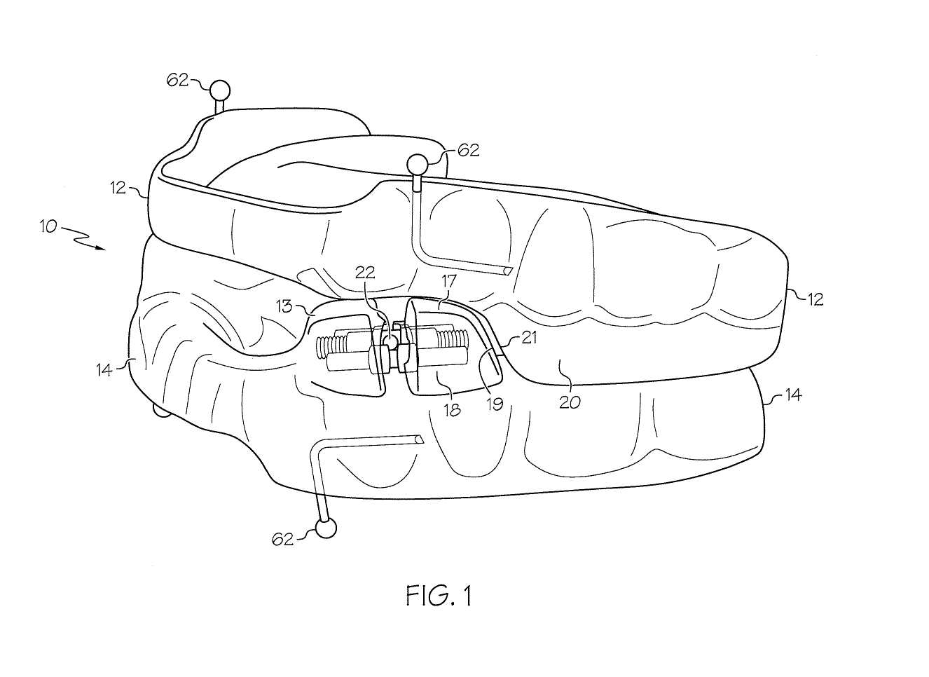

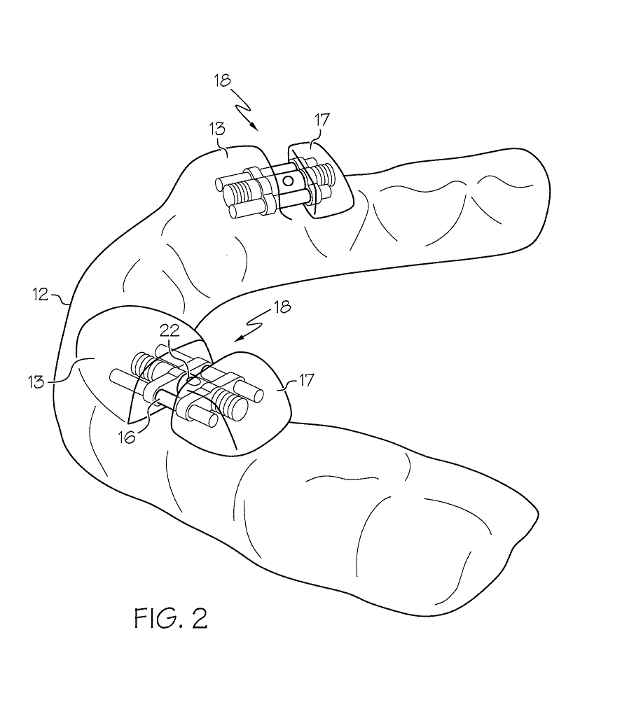

Looking at FIGS. 1 and 2, a first embodiment of the present invention (a “tray” embodiment) provides a removable, adjustable apparatus 10 having an upper plate 12 and a lower plate 14 which together, upon insertion into a user's mouth, promote forward mandibular displacement. The lower plate 14 is shaped to conform to a user's lower dental arch, and includes a pair of adjustable wedges or lower blocks 18 on opposite sides thereof (i.e., one on the right side of the lower dental arch, and another on the left side of the lower dental arch), while the upper plate 12 is shaped to conform to the user's upper dental arch and includes a pair of integral wedges or upper blocks 20 on opposite sides thereof (i.e., one on the right side of the upper dental arch, and another on the left side of the upper dental arch).

Each lower block 18 is a raised block portion of the lower plate 14 that extends upwardly therefrom, and includes a stationary portion 13 and a length-adjustable portion for causing further mandibular advancement, the length-adjustable portion comprising an adjustable advancement screw assembly 16 and a sliding portion 17. Each lower block 18 is typically positioned on the lower plate 14 so that in use it is located over the canine premolar area of the user's lower anterior teeth. The pair of integral wedges or upper blocks 20 of the upper plate, when fitted, is located over the user's second premolar-molar area.

Each sliding portion 17 of the lower plate 14 typically has a downwardly and rearwardly angled contact surface 19 for cooperating with an upwardly and forwardly angled contact surface 21 of its corresponding upper block 20 of the upper plate 12. However, the contact surfaces 19, 21 can have any type of angle as long as the surfaces cooperate with each other to exert opposing forces upon the upper and lower dental arches and cause mandibular advancement. The contact surfaces 19, 21 allow the sliding portion 17 of the lower block 18 to push against the upper block 20, thereby causing the lower jaw to advance forward. The lower and upper blocks 18, 20 thus engage at their contact surfaces 19, 21 in a manner to releasably interlock with and oppose one another when the user's upper jaw and the lower jaw are in a substantially closed position, thereby causing advancement of the lower jaw, i.e. forward mandibular advancement.

When the apparatus 10 is being used while the user is asleep, the relationship between the contact surfaces 19, 21 prevents the user's lower jaw from falling back towards the airway. Looking at FIG. 1, pairs of ball clasps 62 can be embedded in the plates 12, 14 as shown to aid in keeping the upper and lower plates approximated during sleep. This will encourage nasal breathing, and limit mouth breathing. Rubber bands (not shown) can be used to wrap around the ball clasps 62 and prevent the user from opening the mouth too widely during sleep and releasing the interlocking contact surfaces 19, 21 between the upper and lower blocks 20, 18.

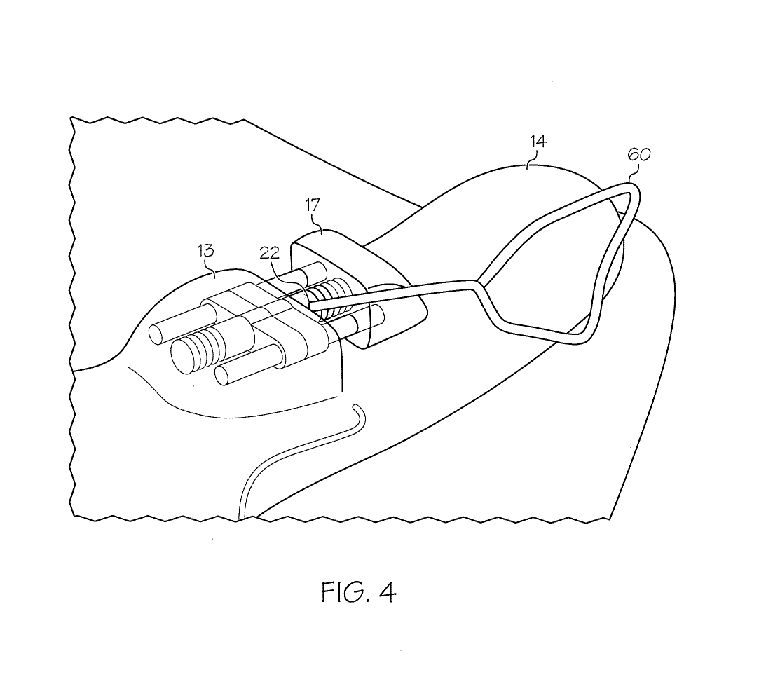

As illustrated in FIG. 2, each of the lower blocks 18 is length-adjustable and includes a stationary portion 13, an adjustable advancement screw 16 with a rotatable advancement arm 22, and a sliding portion 17 connected to the distal end of the advancement arm 22. Rotation of the advancement arm lengthens the length-adjustable block by distally moving the sliding portion 17. The contact between the lower blocks 18 and upper blocks 20 can therefore impart an adjustable amount of mandibular advancement, which can be increased or decreased by way of activating rotatable advancement arms 22, which are part of the adjustable advancement screw assemblies 16. These commercially available expansion screw assemblies 16 are typically marked with an arrow indicating the opening direction. Turning of the rotatable advancement arm 22 will lengthen the advancement screw 16 in order to cause the sliding portion 17 to move and push against the upper block 20 and advance/protrude the lower jaw forward. Advancement is done with the aid of a small straight thin rod or “key” 60, supplied by the manufacturer of the advancement screw assembly, to turn each advancement screw assembly. As shown in FIG. 4, the key 60 fits into (typically one of two to four) holes of the advancement arms 22 to allow the user to rotate the advancement arms. Typically four turns of the key 60 (moving the advancement arm 22 ninety degrees)(90° each turn or 360 degrees after four turns) advances the lower plate 1 mm anteriorly relative to the upper plate.

Each of the advancement screws 16 is a conventional and commercially available product, which functions essentially as a turnbuckle. An advancement arm 22 has guide pins on each side which serve to keep the advancement arm in alignment. Each advancement screw 16 is placed in a mold of the patient's teeth and embedded in acrylic or other suitable synthetic material as part of a lower block 18, with one end embedded in the stationary portion 13 and the other end in the sliding portion 17.

The advancement screws 16 are typically made of stainless steel, chromium-nickel alloys and/or nonferrous metals, thereby providing resistance against oral acidity. Each advancement screw assembly 16 is initially placed in a mold of the lower plate 14 at the desired location, and during the molding process a portion thereof is embedded into place in the acrylic portions 13, 17 of the lower block 18, becoming part of the lower block 18 but with the advancement arm 22 being uncovered and unencumbered by the acrylic. In addition to acrylic, the sliding portion 17 may also be made of stainless steel, chromium-nickel alloys and/or nonferrous metals. The advancement screw 16 is designed to allow the lower block 18 to be completely closed during the embedding process, so that the acrylic portion of the lower block 18 does not enter the advancement arm area 22 and possibly cause jamming.

When in contact with the upper block 20 of the upper plate, lengthening of the advancement screws 16 advances the sliding block 17 forward (anteriorly) and causes forward advancement of the lower plate 14 relative to the upper plate 12, which causes the mandible to incrementally advance forward. Repositioning of the mandible in a more anterior (protrusive) position in this manner, as compared to the normal closed position of the jaw, can help relieve upper airway restrictions behind the oral cavity. Advancement of the mandible also helps to advance the tongue forward in the mouth and reduce the likelihood of the tongue blocking or partially blocking the airway. The apparatus 10 can thus serve to establish a new working posture for the lower jaw and tongue, and the rotatable advancement arms 22 allow the mandible to be gradually advanced to a position of maximal comfortable protrusion, which is associated with optimal reduction in obstructive sleep apnea symptoms such as snoring and apneic events.

The upper and lower plates 12, 14 are typically made out of a suitable synthetic plastic material that is flexible and durable, such as acrylic polymer or other dual laminate material as is known in the art. The lower plate 14 engages the upper plate 12 when inserted by a user and the upper and lower mouth are in a substantially closed position, such that the upper and lower plates 12, 14 act to cause a physical barrier to closing the upper and lower teeth together. This contact between plates increases the vertical distance of the oral cavity, thereby improving airway patency and allowing more room for the tongue to move. Further, during use the plates 12, 14 typically do not disengage, and contact between the contact surfaces 19, 21 of the plates results in advancement of the lower jaw relative to the upper jaw, causing the mandible to advance forward. By doing so, an appropriate volume of the tongue is positioned forward in the oral cavity to relieve upper airway restrictions behind the oral cavity.

The plates 12, 14 therefore serve to automatically reposition the mandible in a more anterior (protrusive) position as compared to the normal closed position of the jaw. Unlike other adjustable mandibular advancement devices, the mechanism of advancement (i.e. the advancement arm 22) is on the occlusal surface (bite side) aspect of the device rather than the outside surface of the teeth or the front of the dental arches. The occlusal surface area has previously been deemed “dead space” since material for fabrication of the plates has to cover this area to attach to the teeth for retention of the device in the user's mouth. By utilizing the occlusal surface to house the advancement arm 22, an anterior (forward) position of the lower jaw can be maintained without causing irritation to the cheeks or the tongue. This will also provide comfort and better compliance with oral appliance therapy for snoring or sleep apnea conditions.

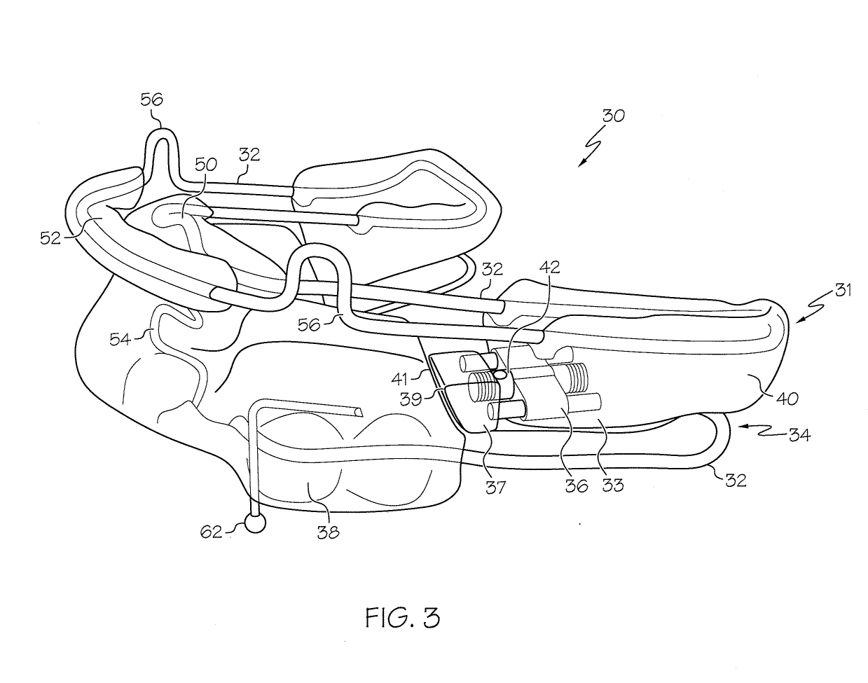

FIG. 3 illustrates another embodiment 30 of the removable, adjustable apparatus of the present invention in which reinforcement or orthodontic wire 32 is used in both an upper plate 31 and lower plate 34 for stabilization. Each plate 31, 34 generally follows the shape of the user's dental arch, and, like the first embodiment discussed above, is manufactured to include an acrylic or dual laminate material that is adaptable to conform to a user's maxillary or mandibular dentition.

In this “wiring” embodiment 30, the upper plate 31 includes a pair of length-adjustable upper blocks 40 extending downwardly from the wiring 32 of the upper plate 31, each upper block 40 having a maxillary advancement screw 36 embedded therein to allow a sliding portion 37 to adjust the length of the upper block 40. Each upper block 40 is typically positioned so that in use it is located over the user's upper second premolar-molar area. The lower plate 34 is also provided with a pair of integral wedges or lower blocks 38 (non-length-adjustable) which, when the plate is fitted, are located over the canine first premolar area of the user's lower anterior teeth.

It is noteworthy that in FIG. 3 the advancement screws 36 and sliding portion 37 are included in the upper blocks 40 in the “wiring” embodiment 30, rather than being in the lower blocks as illustrated in the “tray” embodiment 10 of FIG. 1. Thus, the present invention can include various related embodiments, in which the device can be either a “tray” embodiment (having no orthodontic wiring included) or a “wiring” embodiment, and also in which either the lower blocks or the upper blocks are manufactured as the length-adjustable blocks. Whether or not a tray embodiment (FIGS. 1 and 2) or a wiring embodiment (FIGS. 3 and 4) is used, or a combination thereof in which a tray is used on one arch and wiring on the other, and whether or not the length-adjustable block is placed on the upper or lower plate of the device, depends upon the specific clinical situation presented by the user, and which the dental professional deems is the most advantageous type (tray or wiring embodiment) and location (upper or lower length-adjustable blocks) for that user. For example, if a patient presents with a severe angulation of their teeth, or missing teeth, then the clinician or dental professional can consider using the wiring embodiment in one arch and the tray embodiment in the other.

Further depending on the clinical situation presented by the patient, the upper and lower blocks can also be manufactured in a fashion in which the only contact between the upper and lower plates is the contact between the raised blocks. For example, in FIG. 1 it can be appreciated that the upper block 20 makes contact with both the lower block 17 and the non-block portion of the lower plate 14; however, if a patient presents as a “clencher” or “teeth grinder” then the upper block 20 can be made “smaller” than usual, such that there is a gap between the upper block 20 and the lower plate 14. This will prevent the user from recruiting their temporalis muscles used for teeth grinding, allowing the user to awake from sleep without their typical tension-type headache caused by a night of teeth clenching and grinding.

Looking at FIG. 3, each upper block 40 includes a stationary portion 33, an adjustable advancement screw 36 with an advancement arm 42, and a sliding portion 37 connected to the distal end of the advancement arm 42. Each sliding portion 37 typically has an upwardly and forwardly angled contact surface 39 for cooperating with a corresponding downwardly and rearwardly angled contact surface 41 of one of two lower blocks 38 of the lower plate 34. However, the contact surfaces 39, 41 can have any type of angle as long as the surfaces cooperate with each other to exert opposing forces upon the upper and lower dental arches and cause mandibular advancement. The contact surfaces 39, 41 allow the sliding portion 37 of the upper block 40 to push against the lower block 38. The lower and upper blocks 38, 40 thus engage at their contact surfaces 41, 39 in a manner to releasably interlock with one another when the user's upper jaw and the lower jaw are in a substantially closed position, thereby causing advancement of the lower jaw, i.e. forward mandibular advancement.

The amount of mandibular advancement provided by the contact between the upper blocks 40 and lower blocks 38 can be increased or decreased by way of activating the rotatable advancement arms 42 of the advancement screws 36. Turning the rotatable advancement arm 42 will lengthen the advancement screw 36 in order to cause the sliding portion 37 of the upper block 40 to move and push against the lower block 38 and further advance/protrude the lower jaw forward. Advancement is done as described above for the embodiment of FIGS. 1 and 2, with the aid of a small straight thin rod or “key” 60 supplied by the manufacturer of the advancement screw assembly, to turn each advancement screw assembly (see FIG. 4). The advancement screws 36 are essentially the same as screws 16 in FIGS. 1 and 2.

In addition to the synthetic plastic material such as acrylic polymer or other dual laminate material which make up the blocks 38, 40 of the embodiment 30 in FIG. 3, typically the orthodontic wire 32 has acrylic molded over certain sections, e.g. to provide arched support regions 50, 52, 54 at the front of both the upper and lower plates 31, 33 to hold and protect the upper and lower front teeth of the user. Specifically, to support the upper front teeth the upper plate 31 includes an acrylic-molded front wire 52 and back wire 50, and the lower portion 33 includes an acrylic-molded wire 54 as well. This helps prevent any teeth movement that may possibly occur, and protects the teeth from constant contact with the orthodontic wire. A pair of loops 56 is typically placed just lateral to the acrylic-molded front wire 52 to help loosen or tighten the retention of the apparatus against the front teeth. Laser welding can be used in areas where orthodontic wire is doubled together.

When the apparatus 30 is being used while the user is asleep, the relationship between the contact surfaces 39, 41 prevents the user's lower jaw from falling back towards the airway. A pair of ball clasps 62 can be embedded in the lower plate 34 as shown, and rubber bands (not shown) can be used to wrap around the ball clasps 62 of the lower plate and the loops 56 of the upper plate to prevent the user from opening their mouth too widely, thus preventing release of the interlocking contact surfaces 39, 41 between the upper and lower blocks 40, 38 and to aid in keeping the upper and lower plates 31, 34 approximated during sleep. This will encourage nasal breathing, and limit mouth breathing.

The orthodontic wiring 32 shown in the embodiment of FIG. 3 serves as reinforcement for the plates 31, 34, and is intended to be moldable and bendable. The wiring 32 is not only used to assist in stabilization of the apparatus and retention in the user's mouth, but also to facilitate the user's teeth position in the arch and minimize any tooth movement that may occur. Wiring 32 is typically constructed of stainless steel metal wires having a diameter of between about 0.035 mm to about 1.45 mm, and preferably between about 0.035 mm to about 0.055 mm. The wiring provides form for the plates so they can function properly, as well as stabilize, strengthen and shape the apparatus. All of the wiring is typically stiff enough to maintain the acrylic supports in a stable formation, yet malleable enough for adjustment to fit each individual patient, so that the orthodontist or dentist can re-shape the apparatus until airflow resistance is reduced in the mouth and pharynx and a patent airway is achieved for each individual patient.

The rotatable advancement arms 22, 42 of the different embodiments of the inventive apparatus 10, 30, include keyholes that are placed on the biting (occlusal) side of the user's teeth, and unlike other prior art devices the advancement arms 22, 42 are not located on the side of the apparatus between the teeth and the cheek, or in the front of the apparatus. Therefore, the inventive apparatus is less bulky than other similar prior art devices, typically does not cause soreness in the mouth, and requires little if any intraoral space, making user compliance more likely. Devices such as the Herbst mandibular advancement splint use a rod that attaches to the upper molar teeth on the cheek side and a lower attachment on the premolar teeth. This device may cause irritation of the cheek when the subject is a side sleeper. Additionally it may feel bulky to the subject due to a small oral cavity space. Adjustable dorsal type appliances available to date also have a similar outcome as described with the Herbst appliances. In many cases getting a dorsal type appliance into one's mouth involves a wide mouth opening that is difficult to achieve in some subjects making its placement difficult. A Thornton Adjustable Positioner or TAP device has an anterior component that locks the upper and lower appliance in place. In large tongues or tongue thrusters, the front part of the tongue can become irritated because of the position of the anterior component. The apparatus of the present invention eliminates these issues.

The apparatus of the invention typically retains a patient's mandible in a protrusive position and can have a number of beneficial uses, including as an early interceptive device to encourage mandibular growth, in the treatment of certain orthodontic problems, in the treatment of certain temporomandibular joint problems, in the management of bruxism, and/or in the treatment of snoring and obstructive sleep apnea. The apparatus is fabricated on a custom, per-patient basis. The components necessary for a preferred embodiment may be offered in “kit” form for fabrication of the appliances in local labs and offices. The preferred method of fabricating and custom-fitting the apparatus to the patient comprises an initial office visit and one or more follow-up visits before the apparatus is ready. The apparatus can be formed from flexible and durable orthodontic materials such as acrylic polymer or other dual laminate material, and the orthodontic wires can be made of stainless steel, cobalt, chromium, gold, silver, platinum or other acceptable materials.

While the present invention has been broadly described and illustrated by the embodiments herein, it is not intended to restrict or in any way limit the scope of the invention or the appended claims to such detail. Additional advantages, variations and/or modifications will be readily apparent to those skilled in the art. The present embodiments are, therefore, to be considered in all respects as illustrative and not restrictive.

What is claimed is:

1. A removable, adjustable intraoral advancement apparatus for the treatment of obstructive sleep apnea and/or snoring, comprising:
a) a lower plate manufactured to conform to a user's lower dental arch, the lower plate including a pair of lower blocks extending upwardly therefrom; and
b) an upper plate manufactured to conform to the user's upper dental arch, the upper plate including a pair of upper blocks extending downwardly therefrom, wherein the upper and lower blocks include contact surfaces which cooperate with each other to exert opposing forces upon the upper and lower dental arches and cause mandibular advancement, and wherein each of either the lower blocks or the upper blocks comprises a stationary portion and a length-adjustable portion for causing further mandibular advancement, the length-adjustable portions being located on the occlusal side of the user's teeth.

2. The apparatus of claim 1, wherein each of the lower blocks comprise a stationary portion and a length-adjustable portion.

3. The apparatus of claim 1, wherein each of the upper blocks comprise a stationary portion and a length-adjustable portion.

4. The apparatus of claim 1, wherein each of the length-adjustable portions comprises an adjustable advancement screw and a sliding portion.

5. The apparatus of claim 4, wherein each of the advancement screws includes a rotatable advancement arm, the sliding portion connected to the distal end of the advancement arm, wherein rotation of the advancement arm lengthens the length-adjustable portion by distally moving the sliding portion.

6. The apparatus of claim 5, wherein advancement is done with the aid of a key for turning each advancement screw, and wherein the key is inserted into a hole of the advancement arm to allow rotation of the advancement arm.

7. The apparatus of claim 1, wherein a pair of ball clasps are embedded in each of the upper and lower plates, and wherein rubber bands are used to wrap around the ball clasps to prevent the user from opening the mouth and aid in keeping the upper and lower plates approximated during sleep.

8. The apparatus of claim 1, wherein the upper and lower plates include orthodontic wiring, the upper plate including a pair of loops, and wherein a pair of ball clasps are embedded in the lower plate and rubber bands are used to wrap around the ball clasps of the lower plate and the loops of the upper plate to prevent the user from opening the mouth and aid in keeping the upper and lower plates approximated during sleep.

9. A removable, adjustable intraoral advancement apparatus for managing snoring and obstructive sleep apnea, comprising:
a) a lower plate manufactured to conform to a user's lower dental arch, the lower plate including a pair of lower blocks extending upwardly therefrom, each lower block being adjustable in length and including a stationary portion and a length-adjustable portion; and
b) an upper plate manufactured to conform to the user's upper dental arch, the upper plate including a pair of upper blocks extending downwardly therefrom, wherein the upper and lower blocks include contact surfaces which cooperate with each other to exert opposing forces upon the upper and lower dental arches and cause mandibular advancement, and wherein the length-adjustable portions allow for further mandibular advancement, the length-adjustable portions being located on the occlusal side of the user's teeth.

10. The apparatus of claim 9, wherein each of the length-adjustable portions comprises an adjustable advancement screw and a sliding portion.

11. The apparatus of claim 10, wherein each of the advancement screws includes a rotatable advancement arm, the sliding portion connected to the distal end of the advancement arm, wherein rotation of the advancement arm lengthens the length-adjustable portion by distally moving the sliding portion.

12. The apparatus of claim 11, wherein advancement is done with the aid of a key for turning each advancement screw, and wherein the key is inserted into a hole of the advancement arm to allow rotation of the advancement arm.

13. The apparatus of claim 9, wherein a pair of ball clasps are embedded in each of the upper and lower plates, and wherein rubber bands are used to wrap around the ball clasps to prevent the user from opening the mouth and aid in keeping the upper and lower plates approximated during sleep.

14. The apparatus of claim 9, wherein the lower blocks are not adjustable in length, and wherein each of the upper blocks are adjustable in length and include a stationary portion and a length-adjustable portion.

15. A removable, adjustable intraoral advancement apparatus for managing snoring and obstructive sleep apnea, comprising:
a) a lower plate manufactured to conform to a user's lower dental arch, the lower plate including orthodontic wiring and a pair of lower blocks extending upwardly therefrom;
b) an upper plate manufactured to conform to the user's upper dental arch, the upper plate including orthodontic wiring and a pair of upper blocks extending downwardly therefrom, each upper block being adjustable in length and including a stationary portion and a length-adjustable portion, wherein the upper and lower blocks include contact surfaces which cooperate with each other to exert opposing forces upon the upper and lower dental arches and cause mandibular advancement, and wherein the length-adjustable portions allow for further mandibular advancement, the length-adjustable portions being located on the occlusal side of the user's teeth.

16. The apparatus of claim 15, wherein each of the length-adjustable portions comprises an adjustable advancement screw and a sliding portion.

17. The apparatus of claim 16, wherein each of the advancement screws includes a rotatable advancement arm, the sliding portion connected to the distal end of the advancement arm, wherein rotation of the advancement arm lengthens the length-adjustable portion by distally moving the sliding portion.

18. The apparatus of claim 17, wherein advancement is done with the aid of a key for turning each advancement screw, and wherein the key is inserted into a hole of the advancement arm to allow rotation of the advancement arm.

19. The apparatus of claim 15, wherein the upper plate includes a pair of loops and the lower plate includes a pair of ball clasps embedded therein, and wherein rubber bands are used to wrap around the ball clasps of the lower plate and the loops of the upper plate to prevent the user from opening the mouth and aid in keeping the upper and lower plates approximated during sleep.

20. The apparatus of claim 15, wherein the upper blocks are not adjustable in length, and wherein each of the lower blocks are adjustable in length and include a stationary portion and a length-adjustable portion.
